(19) United States
(12) Patent Application Publication
(10) Pub. No.: US 2010/0140131 A1
(43) Pub. Date: Jun. 10, 2010

(54) Production of an Oxaliplatin Mixture and a Container and a Container Set for Said Mixture

(76) Inventors: Michaela Roth, Reichersbeuern (DE); Katrin Meyer-Wülfing, Munchen (DE)

Correspondence Address: LICATA & TYRRELL P.C., 66 E. MAIN STREET, MARLTON, NJ, 08053, US

(21) Appl. No.: 12/063,531

(22) PCT Filed: Aug. 11, 2006
(86) PCT No.: PCT/EP06/07982

§ 371 Date: Mar. 25, 2008

(30) Foreign Application Priority Data
Aug. 11, 2005 (DE) 102005038347.5

(51) Int. Cl. B65D 85/00; A61K 31/282; A61P 35/00

(52) U.S. Cl. 206/525; 514/492

(57) ABSTRACT

The invention relates to a process for the preparation of an aqueous oxaliplatin solution in which oxaliplatin is dissolved in water by addition of an acid up to an oxaliplatin concentration greater than an acid-free aqueous oxaliplatin solution. The invention relates also to a container and a set of containers containing the solution.

EP 0 943 331 B1 describes a stable oxaliplatin solution containing oxalic acid or an oxalic acid salt as buffer. The solution can be introduced into an ampoule, a glass vial (page 8, line 10), an infusion pouch or a syringe. A disadvantage of that formulation is a certain toxicity of the oxalic acid.

WO 03/047 587 discloses a stable oxaliplatin solution in suitable containers (page 12, line 28) containing lactic acid or a lactic acid salt as buffer.

US 2003/0 109 515 A1 describes a stable oxaliplatin solution in suitable containers (paragraph number [0060]) containing malonic acid or a malonic acid salt as buffer.

It has been found, surprisingly, that the concentration of an aqueous oxaliplatin solution containing an acid can be adjusted to a value greater than the concentration of an acid-free solution.

Accordingly, in accordance with one embodiment the present invention relates to the use of an acid to increase the solubility of oxaliplatin in an aqueous solution.

For example, in accordance with a further embodiment the invention relates to a process for the preparation of an aqueous oxaliplatin solution in which oxaliplatin is dissolved in water by addition of an acid up to an oxaliplatin concentration greater than an acid-free aqueous oxaliplatin solution, especially under conditions that are otherwise the same.

Furthermore, the present invention relates to a process for the preparation of a solution consisting of oxaliplatin, an acid and water, in which oxaliplatin is dissolved in water by addition of an acid up to an oxaliplatin concentration greater than an acid-free aqueous oxaliplatin solution.

In the process according to the invention, oxaliplatin may be dissolved in water by addition of the acid up to the highest oxaliplatin saturation concentration achievable with the aid of that acid.

Furthermore, in the process according to the invention an oxaliplatin concentration can be stipulated that lies in the range defined on the one hand by the saturation concentration of an acid-free aqueous oxaliplatin solution and on the other hand by the highest saturation concentration in the presence of the acid, and the acid is added until the stipulated concentration has been achieved.

In accordance with a preferred embodiment, in the process of the present invention no carbohydrate, such as, for example, lactose, glucose, maltose, fructose, galactose or dextrans (e.g. 10-70), is added to the solution.

In accordance with a further preferred embodiment, in the process of the present invention no polyethylene glycol, such as, for example, polyethylene glycol 200, 300, 400 or 600, is added to the solution.

Furthermore, in the process according to the invention it is possible to establish an oxaliplatin concentration that is a whole-numbered value or half-numbered value (whole number plus ½) of a customary concentration measurement unit, the measurement unit preferably being mg/ml or molar.

Furthermore, in the process according to the invention an inorganic and/or organic acid can be used.

For example, at least one inorganic acid from the group formed by sulfuric acid, nitric acid and phosphoric acid can be used. Preference is given to the use of sulfuric acid.

Furthermore, at least one organic acid from the group formed by citric acid, succinic acid, ascorbic acid, oxalic acid, lactic acid and malonic acid can be used. Preference is given to the use of citric acid.

Furthermore, in the process according to the invention a buffering salt may additionally be used.

Furthermore, in the process according to the invention a pH value of from 1 to 7 and especially from 1.5 to 4 may be established.

Furthermore, in the process according to the invention the prepared solution can be introduced into a container and the container closed.

Furthermore, in the process according to the invention the container can be closed under an inert gas.

Furthermore, in the process according to the invention an injection bottle (vial), a screw closure bottle or an ampoule can be provided as container.

Furthermore, in the process according to the invention an injection bottle, in the form of a single dose or multiple dose container, can be used as bottle.

A further embodiment of the invention relates to a container containing an aqueous oxaliplatin solution, obtainable in accordance with the process of the invention.

A further embodiment of the invention relates to a container containing an acidic aqueous oxaliplatin solution having an oxaliplatin concentration greater than an acid-free aqueous oxaliplatin solution.

A further embodiment of the invention relates to a container containing an acidic aqueous oxaliplatin solution having the highest oxaliplatin saturation concentration achievable with the aid of that acid or having the highest oxaliplatin saturation concentration that is characteristic of the acid in question.

A further embodiment of the invention relates to a container containing an acidic aqueous oxaliplatin solution having an oxaliplatin concentration that is a whole-numbered value or half-numbered value (whole number plus ½) of a customary concentration measurement unit, the measurement unit preferably being mg oxaliplatin/ml solution or mg oxaliplatin/mg solution or molar.

A further embodiment of the invention relates to a set of containers or a set comprising containers according to the invention each containing an acidic aqueous oxaliplatin solution having an oxaliplatin concentration that is a whole-numbered value or half-numbered value (whole number plus ½) of a customary concentration measurement unit, the measurement unit preferably being mg oxaliplatin/ml solution or mg oxaliplatin/mg solution or molar.

Finally, an embodiment of the invention relates to a set of containers or a set comprising containers according to the invention each containing an acidic aqueous oxaliplatin solution having an oxaliplatin concentration that is a whole-numbered value or half-numbered value (whole number plus ½) of a customary concentration measurement unit, the measurement unit preferably being mg oxaliplatin/ml solution or mg oxaliplatin/mg solution or molar, the oxaliplatin concentration of at least one container being different from the concentration of the other container(s).

The present invention also includes the solutions prepared in accordance with one of the described processes.

The increased oxaliplatin solubility in the presence of acid is described in tabular form below with reference to 5 examples.

| | Water | H₂SO₄ | H₃PO₄ | Citric acid | Lactic acid |
|---|---|---|---|---|---|
| pH | 4.4 | 3.5 | 1.6 | 2.1 | 3.5 |
| Molarity | | 0.0025 | 0.08 | 0.1 | |
| of the acid | | | | | |
| Solubility | 5.7 | 6.0 | 6.2 | 7.35 | 6.8 |
| [mg/ml] | | | | | |

1: Process for the preparation of an aqueous oxaliplatin solution in which oxaliplatin is dissolved in water by addition of an acid up to an oxaliplatin concentration greater than an acid-free oxaliplatin solution.

2: Process according to claim 1 for the preparation of a solution consisting of oxaliplatin, an acid and water, in which oxaliplatin is dissolved in water by addition of an acid up to an oxaliplatin concentration greater than an acid-free aqueous oxaliplatin solution.

3: Process according to claim 1 in which oxaliplatin is dissolved in water by addition of the acid up to the highest oxaliplatin saturation concentration achievable with the aid of that acid.

4: Process according to claim 1 in which oxaliplatin concentration is stipulated that lies in the range between the saturation concentration of an acid-free aqueous oxaliplatin solution and the highest saturation concentration in the presence of the acid, and the acid is added until the stipulated concentration has been achieved.

5: Process according to claim 1, characterised in that no carbohydrate is added to the solution.

6: Process according to claim 5, in which an oxaliplatin concentration is established that is a whole-numbered value or half-numbered value of a customary concentration measurement unit, the measurement unit preferably being mg/ml or molar.

7: Process according to claim 1, in which an inorganic and/or organic acid is used.

8: Process according to claim 7, in which at least one inorganic acid selected from the group consisting of sulfuric acid, nitric acid and phosphoric acid is used.

9: Process according to claim 7, in which at least one organic acid selected from the group consisting of citric acid, succinic acid, ascorbic acid, oxalic acid, lactic acid and malonic acid is used.

10: Process according to claim 1, in which a buffering agent is additionally used.

11: Process according to claim 7, in which a pH value of from 1 to 7 is established.

12: Process according to claim 1, in which the prepared solution is introduced into a container and the container is closed.

13: Process according to claim 12, in which the container is closed under an inert gas.

14: Process according to claim 12, in which an injection bottle or vial, a screw closure bottle or an ampoule is provided as container.

15: Process according to claim 14, there being used as bottle an injection bottle in the form of a single dose or multiple dose container.

16: Container containing aqueous oxaliplatin solution, obtainable by a process according to claim 1.

17: Container containing an acidic aqueous oxaliplatin solution having an oxaliplatin concentration greater than an acid-free aqueous oxaliplatin solution.

18. Container according to claim 17 containing an acidic aqueous oxaliplatin solution having the highest oxaliplatin saturation concentration achievable with the aid of a selected acid or having the highest oxaliplatin saturation concentration that is characteristic of the selected acid.

19. Container according to claim 17 containing an acidic aqueous oxaliplatin solution having an oxaliplatin concentration that is a whole-numbered value or half-numbered value of a customary concentration measurement unit, the measurement unit preferably being mg oxaliplatin/ml solution or mg oxaliplatin/mg solution or molar.

20; Set of containers or set comprising. containers according to claim 19 each containing an acidic aqueous oxaliplatin solution having an oxaliplatin concentration that is a whole-numbered value or a half-numbered value of a customary concentration measurement unit, the measurement unit preferably being mg oxaliplatin/ml solution or mg oxaliplatin/mg solution or molar.

21: Set of containers or set comprising containers according to claim 17 each containing an acidic aqueous oxaliplatin solution having an oxaliplatin concentration that is a whole-numbered value or half-numbered value of a customary concentration measurement unit, the measurement unit preferably being mg oxaliplatin/ml solution or mg oxaliplatin/mg solution or molar, the oxaliplatin concentration of at least one container being different from the concentration of the other container.

22: Aqueous oxaliplatin solution, preparable by a process according to claim 1.

23: Use of an acid to increase the solubility of oxaliplatin in an aqueous solution.

24: Process according to claim 11 in which a pH value of from 1.5 to 4 is established.
